(19) United States
(12) Patent Application Publication
(10) Pub. No.: US 2024/0115880 A1
(43) Pub. Date: Apr. 11, 2024

(54) APPLICATORS, SYSTEMS, DEVICES AND METHODS FOR CERVICAL BRACHYTHERAPY

(71) Applicant: Maison Amori oqvpo LLC, New York, NY (US)

(72) Inventor: Nicola NASSER, New York, NY (US)

(21) Appl. No.: 18/545,011

(22) Filed: Dec. 19, 2023

Related U.S. Application Data
(63) Continuation of application No. PCT/US2022/035183, filed on Jun. 27, 2022.
(60) Provisional application No. 63/216,160, filed on Jun. 29, 2021.

(51) Int. Cl. A61N 5/10; A61F 6/14

(52) U.S. Cl. CPC A61N 5/1016; A61F 6/144; A61N 2005/1003; A61N 2005/1008; A61N 2005/1092

(57) ABSTRACT

Exemplary applicator(s), devices, systems and methods can be provided for delivering high dose rate radiation therapy, e.g., to patients with cervical cancer. For example, an intrauterine channel can be provided for or with such applicator(s), system(s), device(s) and methods. Further, at least a part of the device should be fixable within the uterus, and be configured and sized to be able to easily inserted and/or removed from the uterus, while be able to easily affix the exemplary device, applicator, etc. in the cavity of the uterus, and hold the channel anchored to the uterus, as it is larger from the cervical canal.

### CROSS-REFERENCE TO RELATED APPLICATION(S)

This application is a continuation of International Patent Application No. PCT/US2022/035183, filed on Jun. 27, 2022 that published as International Patent Publication No. WO 2023/278357 on Jan. 5, 2023, and also relates to and claims priority from U.S. Provisional Patent Application No. 63/216,160, filed on Jun. 29, 2021, the entire disclosures of which are incorporated herein by reference.

### FIELD OF THE DISCLOSURE

The present application relates to brachytherapy, and more particularly to applicators and methods for cervical brachytherapy.

### BACKGROUND INFORMATION

Cervical cancer took the lives of 4138 women in the USA in 2018; this is the equivalent of 11 women per day, one-half of whom were aged ≤58 years at death. It also continues to be the second leading cause of cancer death in women aged 20 to 39 years [1]. Cervical cancer death rates, are 2 times higher in high-poverty versus low-poverty areas [1]. Cervical cancer incidence and mortality currently vary 2-fold to 3-fold, with incidence rates ranging from <5 per 100,000 in Vermont and New Hampshire, to 10 per 100,000 in Arkansas and Kentucky, and 13 per 100,000 in Puerto Rico, and 7.6 per 100,000 on average for the United States [1]. The American Cancer Society's estimates that 14,480 new cases of cervical cancer will be diagnosed in the United States and about 4,290 women will die from it during 2021 [2]. Worldwide cervical cancer is the fourth most common malignancy in women, with an estimated 570,000 women were diagnosed with cervical cancer in 2018 worldwide and about 311,000 women died from the disease [3].

While a very early-stage cervical cancer can be addressed and/or cured with surgery, locally advanced disease needs definitive treatment with chemotherapy and radiation. Radiation therapy for cervical cancer in the definitive setting is delivered by combining external beam radiation, and brachytherapy. Brachytherapy can provide very high doses of radiation to the cervix and uterus, while sparing nearby organs from radiation and is crucial to cure of this disease.

Brachytherapy for cervical cancer is a procedure that is performed under a general or spinal anesthesia. After cleaning the genitalia, the cervix is grabbed with a tenaculum, and serial dilatations are performed starting with a thin, low Hegar number rod—progressing gradually to larger numbers. At the end of the process, a Smith Sleeve is inserted into the cervical canal, and stitched to it.

After the patient recovers, she comes to 4-5 sessions of treatment during which tandem is inserted into the smith sleeve to facilitate a delivery of radiation into the tandem channel, and ovoids or ring are introduced to treat the cervix and tissues immediately near it.

While brachytherapy using current applicators can be highly effective, such procedure can be very painful to the patients. A surgical dilatation of the cervix should be performed in an operative room, with a high expertise in the procedure to prevent rupture of the uterus, and time and experience for stitching the cervical smith sleeve. The treatment days, in which the insertion of tandem and ovoids are done, are usually performed with pain killers or sedation, but without general or local anesthesia—a procedure that is associated with discomfort and pain, in many instances.

While the applicators that are currently used to treat cervical cancer were designed for the two-dimensional imaging era—that historically used low dose rate brachytherapy, same applicators are still used for the three-dimensional (3D) planning using imaging such as computed tomography (CT) scanning, and magnetic resonance imaging (MRI) while utilizing a high dose rate brachytherapy that uses a source with very small dimensions.

When the current applicators are used, the patient may not be able to stand or walk, and should be in bed while the applicator is bulging out of the vagina, throughout the process of imaging, planning and treatment, which could take few hours.

Thus, there is a need to provide a miniature applicator, that facilitates the patients to stand, walk and function normally after its insertion, so patients can walk to the MRI suit after the insertion of the device, and then walk to a the HDR room, and be able to function within normal parameters while the device in the uterus between the brachytherapy treatments. To address and/or improve such issues and/or deficiencies which exist in the previous devices and processes, the exemplary embodiments of the present disclosure are described.

### SUMMARY OF EXEMPLARY EMBODIMENTS

To that end, according to an exemplary embodiment of the present disclosure, an exemplary applicator can be provided for delivery of high dose rate brachytherapy for cervical cancer. Such exemplary applicator can be inserted, e.g., bed-side similar to Intrauterine Device (IUD) and vaginal ring, without or with anesthesia. The exemplary applicator facilitates an improved utilization of MRI imaging easier to the patient, and making treatment planning much more accurate.

An exemplary system, applicator and method according to the exemplary embodiments of the present disclosure can be provided, in which the uterine and cervical canal can be accessed by introducing an intrauterine High-Dose Rate (HDR) channel that can be fixed to the uterus, e.g., with T-shaped wings. The exemplary device, applicator or system can be inserted bedside. After the insertion, a cover that houses the wings can be removed, which can facilitate the opening the wings, and attaching the device to the uterus. The HDR channel can exit through the cervix and stay in the vagina. A vaginal ring can then be inserted by the physician or the patient, so that the ring is near or hugs the cervix. The ring can have the HDR channel in it, that is attached, e.g., only to one side of the rubber vaginal ring, this allows the vaginal ring to be stretched without changing the length of the HDR channel in the ring.

The vagina is then packed with wet pads or air balloons to increase the space between the cervix, and the rectum and bladder. Thereafter, imaging can be performed with MRI (preferably) or CT scan. Then, the target volumes are drawn, as well as organs at risk. A brachytherapy plan is then generated. After that, the patient can be taken to the HDR room, the intrauterine HDR channel and vaginal ring HDR channel can be connected to the HDR afterloader, and the treatment delivered.

The applicator, the device and/or the system can be kept in its location for a number of weeks until a completion of 4-5 fractions of brachytherapy, after which it can be removed. Alternatively, the applicator, the device and/or the system can be removed after each fraction. If 3D planning is not available, the exemplary applicator, the exemplary device and/or the exemplary system can be used for a classical planning using point A and point B.

According to another exemplary embodiment of the present disclosure, a further exemplary applicator can be provided to be used for a high dose rate brachytherapy for uterine cancer. For example, such exemplary applicator can include two (or more) intrauterine channels that bends at its uterine end to form wing shapes. The bended parts can be positioned in the uterine horns. The bent parts can be forced into an “inserter” before insertion into the uterus. During the insertion, two (or more) guide wires can be inserted into the two HDR channels. After inserting into the uterus, the inserter can be retracted to facilitate the uterine end of the HDR channels to return to its bent position. The inserter can then be removed totally, then the guide wires are removed totally.

In yet another exemplary embodiment of the present disclosure, a further exemplary applicator can be provided for a high dose rate brachytherapy for uterine cancer. For example, such applicator can include a channel that has an inflation unit. A HDR channel can be inserted inside such channel. The channel with a deflated inflation unit can be inserted into the uterus, and then the inflation unit can be inflated, which can anchor the channel to the uterus. A stopper can then be attached to the outer side of the channel. Then, the HDR channel can be inserted inside the channel, advanced to the uterine fundus, and then locked in its location. Thereafter, a ring with holes for interstitial brachytherapy can be attached to the main HDR channel.

Using the same or similar structure of the high dose rate brachytherapy for uterine cancer, an exemplary intrauterine device for contraception can be provided. The exemplary device can include a hormonal reservoir, and can be made from copper or any other material. This exemplary device can have a thickness of, e.g., about 2 mm, which is about half of the thickness of the current IUDs, making it easier to insert the exemplary device. This device can have an inflatable balloon and/or inflation unit to facilitate the fixation of the device in the uterus.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a front cross-sectional view of an exemplary HDR intrauterine device provided in the uterus, in accordance with the exemplary embodiment of the present disclosure;

FIGS. 2A-2C are side views of an exemplary operation of the exemplary HDR intrauterine device shown in FIG. 1 as if inserted into the uterus in operation;

FIG. 3 is a set of side views of an exemplary operation of the exemplary HDR intrauterine device according to another exemplary embodiment of the present disclosure, as if removed from the uterus;

FIG. 4 is a cross-section view of a vaginal ring provided with a channel, according to an exemplary embodiment of the present disclosure;

FIG. 5 is a cross-section view of a vaginal ring provided with two channels, according to another exemplary embodiment of the present disclosure;

FIG. 6 is a cross-sectional view of an exemplary usage of the HDR intrauterine device with an exemplary IUD HDR applicator part of a channel that being deposited around the cervix and near the parametria, according to an exemplary embodiment of the present disclosure;

FIG. 7A is a side view of an exemplary operation of the HDR intra uterine device with wings being forced within the inserter, according to the exemplary embodiment of the present disclosure;

FIG. 7B is a side view of the exemplary operation of the HDR intra uterine device with the wings starting to be extruded from the inserter by partially removing the inserter, according to the exemplary embodiment of the present disclosure;

FIG. 7C is a side view of the exemplary operation of the HDR intra uterine device which is pushed to the uterine fundus with wings fully extruded from the inserter and opening within the uterus, according to the exemplary embodiment of the present disclosure;

FIGS. 7D and 7E are side views of the exemplary operation of the HDR intra uterine device with the inserter, respectively, partially and/or fully removed from the uterus, according to the exemplary embodiment of the present disclosure;

FIG. 7F is a side view of the exemplary operation of the HDR intra uterine device with a guide wire removed from the uterus, according to the exemplary embodiment of the present disclosure;

FIG. 8A is a side view of the HDR intra uterine device inserted into uterus and in place therein, according to the exemplary embodiment of the present disclosure; with cervical ring is being introduced.

FIG. 8B is a side view of the HDR intra uterine device inserted into uterus and in place therein together with the HDR cervical ring in place, according to the exemplary embodiment of the present disclosure;

FIG. 8C is a side view of the HDR intra uterine device inserted into uterus and in place therein, and connected to the HDR afterloader with HDR channel, together with the HDR cervical ring, according to the exemplary embodiment of the present disclosure;

FIGS. 9A-9D are side views of an exemplary operation of the HDR intra uterine device, according to a further exemplary embodiment of the present disclosure;

FIG. 10A is a side view of the exemplary operation of the HDR intra uterine device shown in FIG. 9 with the wings in a closed configuration when inserted into the uterus, according to an exemplary embodiment of the present disclosure;

FIGS. 10B-10E are side views of the exemplary operation of the HDR intra uterine device shown in FIG. 9 with the wings in a configuration when inserted into the uterus, and the progress of the inserter and the guide wire being removed from the uterus, according to an exemplary embodiment of the present disclosure;

FIG. 11 is a set of side views of the cervical ring, with right and left HDR channels, and provided with or without balloons and interstitial brachytherapy holes, according to still another exemplary embodiment of the present disclosure;

FIG. 12 is a perspective view of a sleeve shaped applicator that includes proximal and distal rings with HDR channels therein, according to a further exemplary embodiment of the present disclosure;

FIG. 13 is a set of views providing a double tandem uterine applicator, and an exemplary operation thereof, according yet another exemplary embodiment of the present disclosure;

FIGS. 14A-14F are views of the uterine applicator that includes channel(s) which has an inflation unit, and an exemplary operation thereof, according yet a further exemplary embodiment of the present disclosure;

FIG. 15A is a set of side views of an exemplary operation of the contraception intrauterine device with inflation unit for fixation (rather than wings) inserted into the uterus, according to the exemplary embodiment of the present disclosure;

FIG. 15B is a side view of the exemplary operation of the contraception intrauterine device inserted into the uterus while the inflation unit is deflated, according to the exemplary embodiment of the present disclosure;

FIG. 15C is a side view of the exemplary operation of the contraception intrauterine device inserted into the uterus and the inflation unit inflated therein, according to the exemplary embodiment of the present disclosure;

FIG. 15D is a side view of the exemplary operation of the contraception intrauterine device inserted into the uterus and the inflation unit inflated therein and the inserter being removed, according to the exemplary embodiment of the present disclosure; and

FIG. 15E is a side view of the exemplary operation of the contraception intrauterine device inserted into the uterus and the inflation unit inflated therein, the inserter removed, and the inflating channel plugged, according to the exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended claims.

### DETAILED DESCRIPTION OF EXEMPLARY EMBODIMENTS

The following description of exemplary embodiments provides non-limiting representative examples referencing numerals to particularly describe features and teachings of different aspects and exemplary embodiments of the present disclosure. The exemplary embodiments described herein should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the invention to such an extent that other implementations, not specifically covered but within the knowledge of a person of skill in the art having read the description of embodiments, would be understood to be consistent with an application of the exemplary embodiments of the present disclosure.

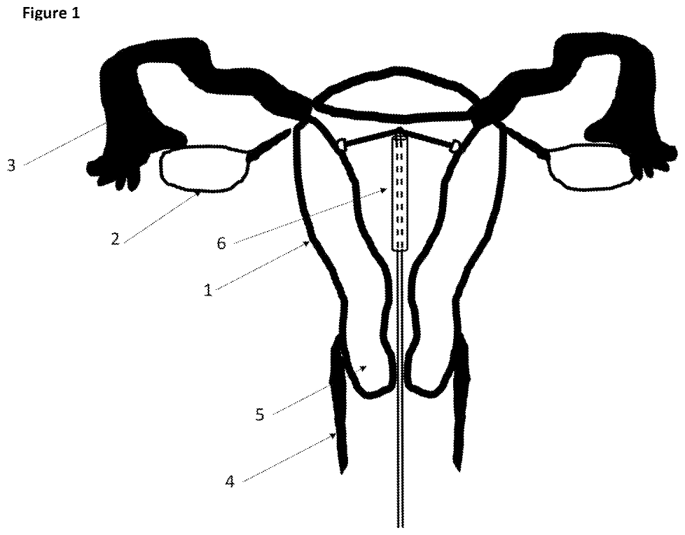

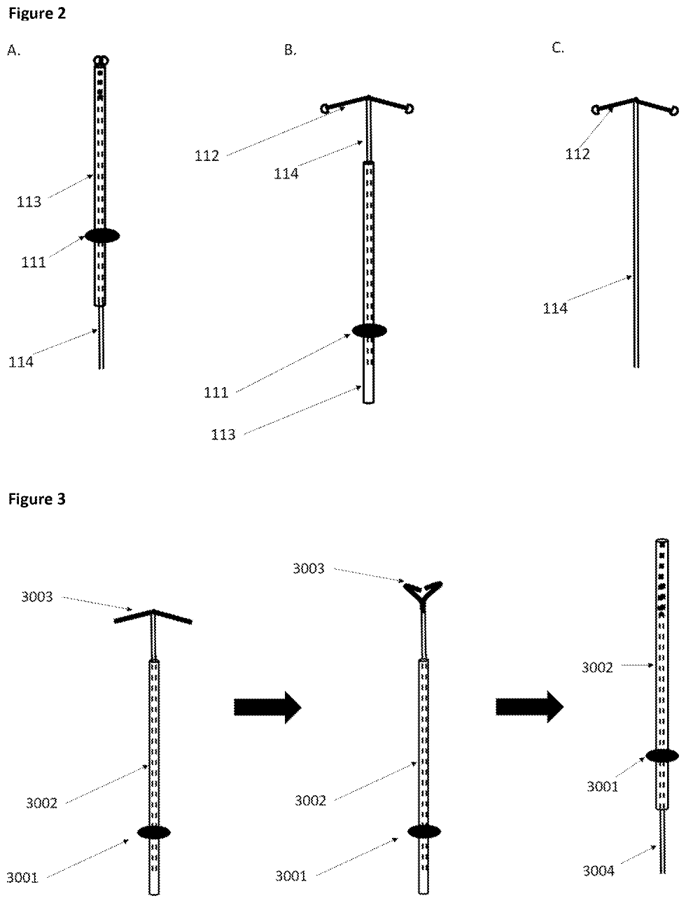

FIG. 1 shows a front cross-sectional view of an exemplary HDR intrauterine device (6) provided in the uterus, in accordance with the exemplary embodiment of the present disclosure. As illustrated in FIG. 1, Uterus (1), Ovary (2), Fallopian tube (3), Vagina (4), and Cervix (5) are provided, and shown in relation of the inserted HDR intra uterine device (6). FIGS. 2A-2C show side views of an exemplary operation of the exemplary HDR intrauterine device shown in FIG. 1 as if inserted into the uterus in operation. For example, FIG. 2A provides the exemplary intrauterine HDR Device in a closed configuration. FIG. 2B shows that a cylindrical housing (113) that keeps the wings in close state, partially removed, and wings (112) open. FIG. 2C shows the exemplary HDR intra uterine device in a fully open state.

FIG. 3 shows a set of side views of an exemplary operation of the exemplary HDR intrauterine device according to another exemplary embodiment of the present disclosure. As shown in FIG. 3, the exemplary HDR intrauterine device can have a stopper (3001), an inserter (3002) and wings (3003). Before the insertion, the wings (3003) are folded into the inserter (3002). After the exemplary HDR intrauterine device is introduced into the uterus, the inserter (3003) is removed thereby allowing unfolding of the wings (3003), which can anchor the exemplary HDR intrauterine device to the uterus, thereby facilitating the delivery of radiation through a HDR channel (3004).

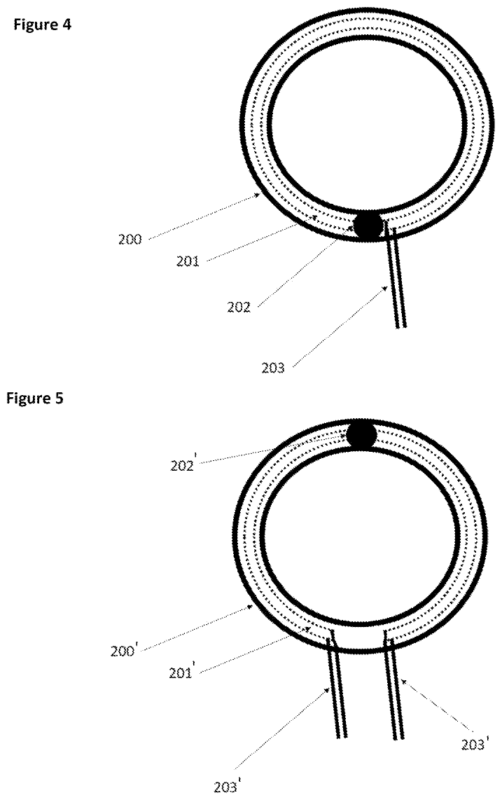

FIG. 4 shows a cross-section view of a vaginal ring (200) provided with a HDR channel (201) and (203), according to an exemplary embodiment of the present disclosure. As provided in FIG. 4, a HDR channel (201) and (203) is provided in the vaginal ring (200) and attached to one side of a rubber vaginal ring anchor (202). This exemplary configuration facilitates the vaginal ring (200) to be stretched without changing the length of the HDR channel (203) in the ring.

FIG. 5 shows a cross-section view of a vaginal ring (200′) provided with two channels (201′) and (203′), according to another exemplary embodiment of the present disclosure. This exemplary configuration of the channels (203′) facilitates an easier motion of the source in the channels (201′) and (203′). The vaginal HDR channels (201′) and (203′) are attached from one side to the vaginal ring anchor (202′), which facilitates the vaginal ring (200′) to be stretched without changing the length of the HDR channels (201′) and (203′).

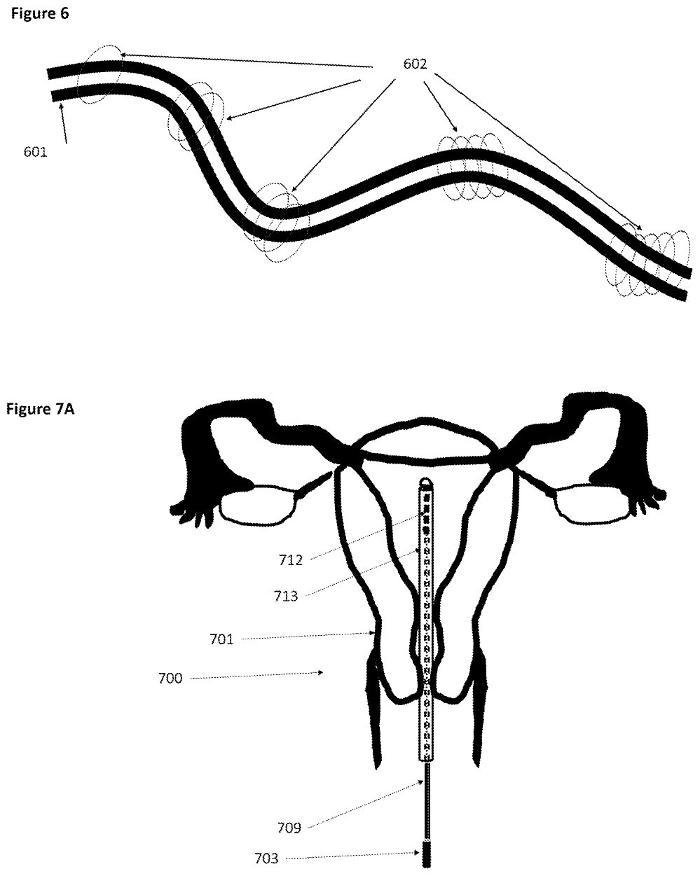

FIG. 6 shows a cross-sectional view of an exemplary usage of the HDR intrauterine device with an exemplary IUD HDR applicator part of a channel that being deposited around the cervix and near the parametria, according to an exemplary embodiment of the present disclosure. As shown in FIG. 6, the exemplary applicator can includes a HDR channel (601) that can have unique markers on specific locations indicating distance between specific numbers of dwells (602). For example, fixed distances can exist between increasing number of circles/markers, that facilitate a visualization of the markers under x ray, CT, MRI or ultrasound, to determine the direction of the channel, and the location of the dwells (602) in relation to the tumor for HDR brachytherapy planning.

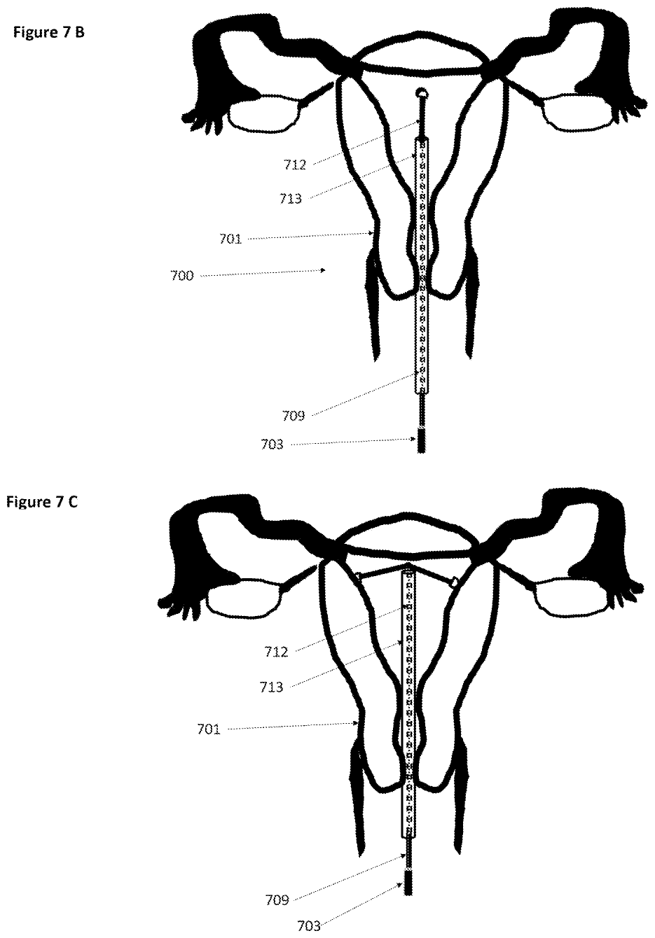

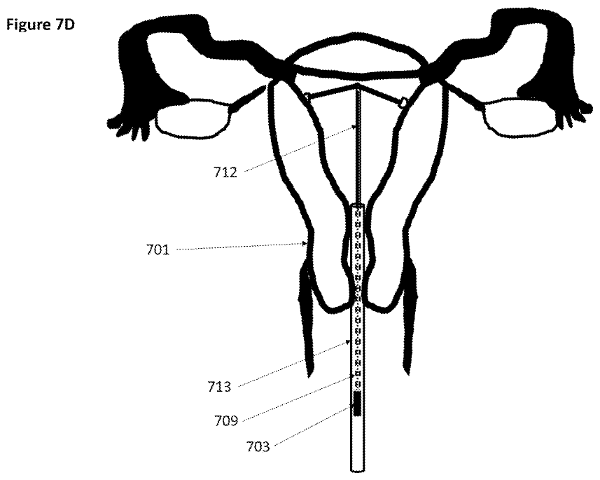

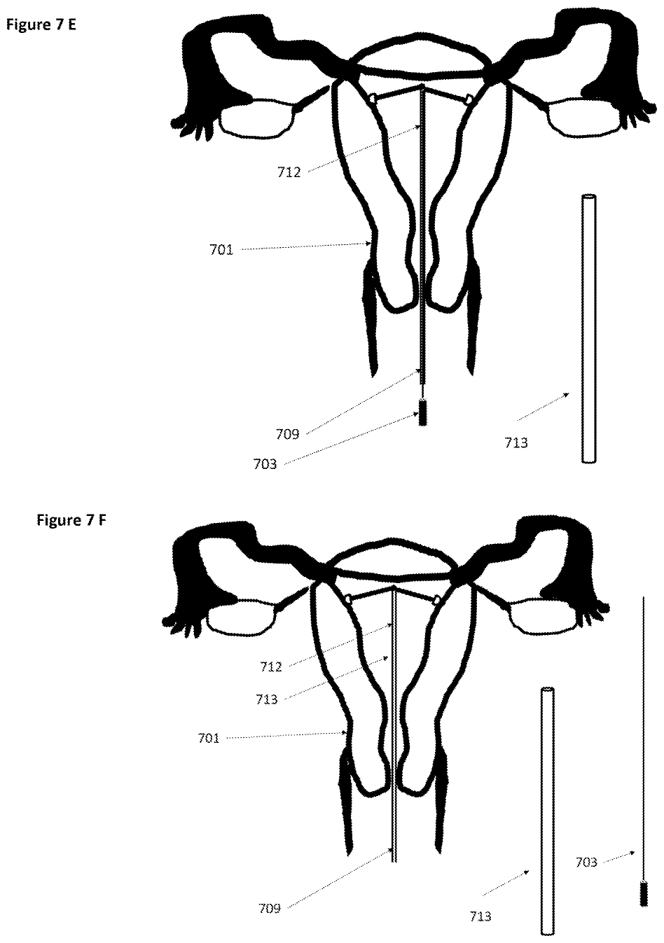

FIG. 7A shows a side view of an exemplary operation of the HDR intra uterine device with wings being forced within an inserter (713), according to the exemplary embodiment of the present disclosure. As shown therein, wings (712) are forced into the inserter (713) and the guidewire (703) is introduced into the HDR channel (709) before the insertion into the uterus (701). FIG. 7B shows a side view of the exemplary operation of the HDR intra uterine device with the wings starting to be extruded from the inserter by partially removing the inserter (713), according to the exemplary embodiment of the present disclosure. In particular, after the insertion into the uterus (701), the inserter (713) which may be cylindrical and that keeps the wings in close state, is partially removed which results in opening of the wings (712).

FIG. 7C shows a side view of the exemplary operation of the HDR intra uterine device which is pushed to the uterine fundus with the wings fully extruded from the inserter (713) and opening within the uterus (701), according to the exemplary embodiment of the present disclosure. FIGS. 7D and 7E illustrate side views of the exemplary operation of the HDR intra uterine device with the inserter (713), respectively, partially and/or fully removed from the uterus (701), and the wings (712) being extended, according to the exemplary embodiment of the present disclosure; FIG. 7F shows a side view of the exemplary operation of the HDR intra uterine device with a guide wire (703) removed from the uterus (701).

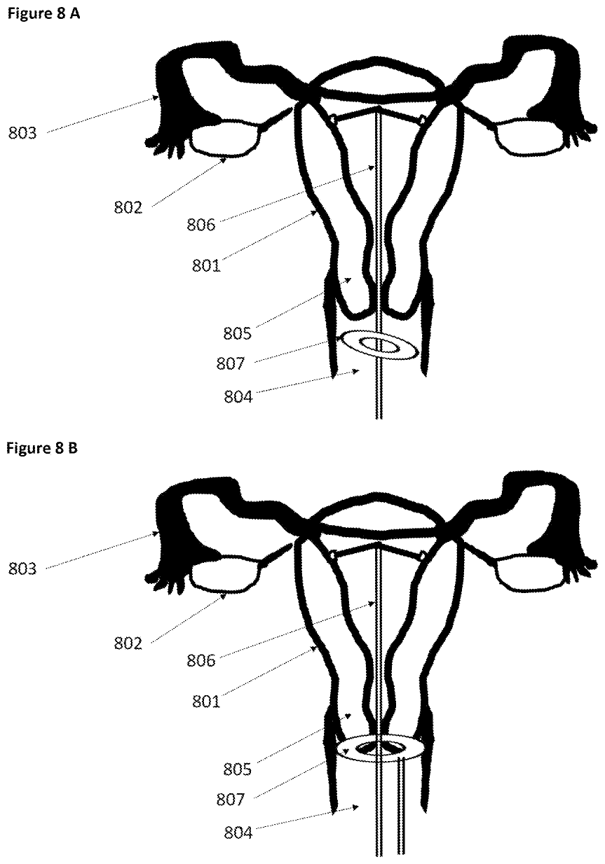

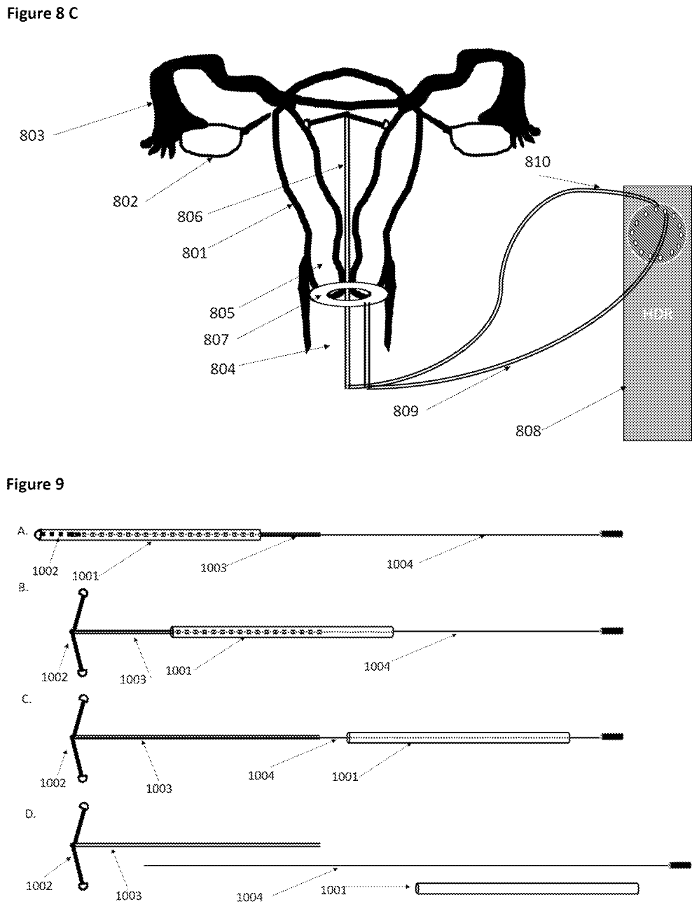

FIG. 8A shows a side view of the HDR intra uterine device (806) inserted into the uterus (801) and in place therein, according to the exemplary embodiment of the present disclosure. Also shown in FIG. 8A are ovaries (802), a fallopian tube (803), a vagina (804), and a cervix (805), with the wings of the HDR intra uterine device (806) being in an extended state, and HDR cervical ring (807) in process of being inserted. FIG. 8B illustrates a side view of the HDR intra uterine device (806) inserted into the uterus (801) and in place therein together with the HDR cervical ring (807), according to the exemplary embodiment of the present disclosure. FIG. 8C shows the HDR intra uterine device (807) already being inserted into the uterus (801) and in place therein, and connected to the HDR afterloader (808) together with the HDR cervical ring (807) being secured around the cervix uteri (805) and being connected to the HDR afterloader (808) with a different HDR channel (809).

FIGS. 9A-9D illustrate side views of an exemplary operation of the HDR intra uterine device, according to a further exemplary embodiment of the present disclosure. Indeed, as shown in FIG. 9A, an exemplary applicator for high dose rate brachytherapy for cervical cancer is provided in FIGS. 9A-9D, that has an intrauterine channel (1003) with wings (1002) that can be closed. FIG. 9A illustrates the wings (1002) being closed, and in FIGS. 9B-9D being opened. Indeed, the wings (1002) can be housed in an “inserter” (1001). As indicated in FIGS. 9A-9C, during the insertion of the HDR intra uterine device, a guide wire (1004) can be inserted into the HDR channel. FIG. 9B shows that after inserting the channel with the wings (1002) housed in the inserter (1001) into the uterus, the inserter (1001) can be retracted to facilitate the release of the wings (1002). Then, as shown in FIG. 9C, the channel is pushed to the uterus fundus by the guide wire (1004). Further, as provided in FIG. 9D, the inserter (1001) and the guide wire (1004) are then completely removed.

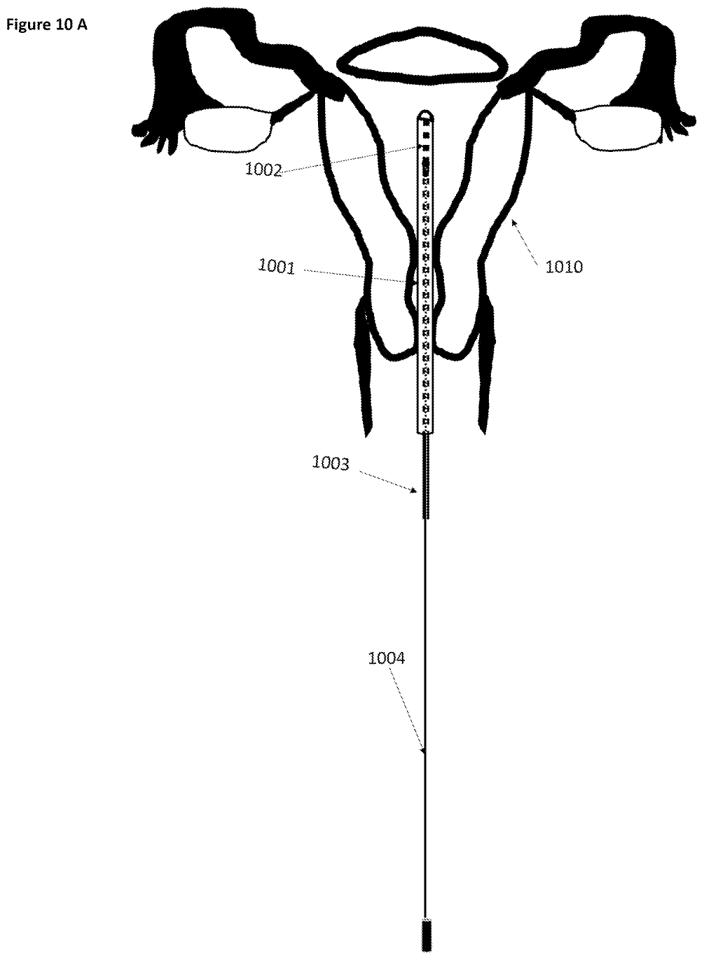

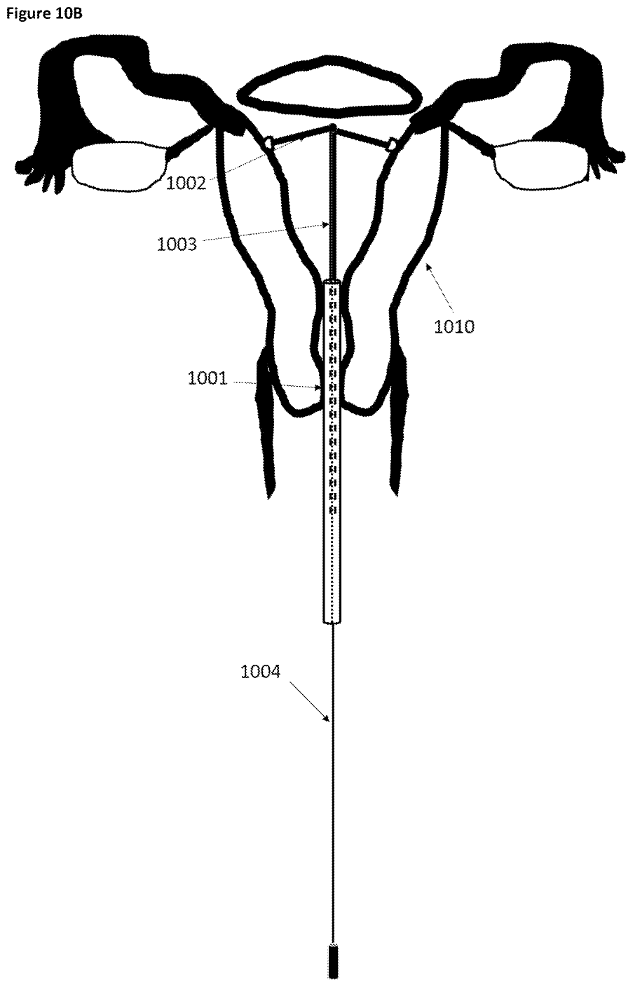

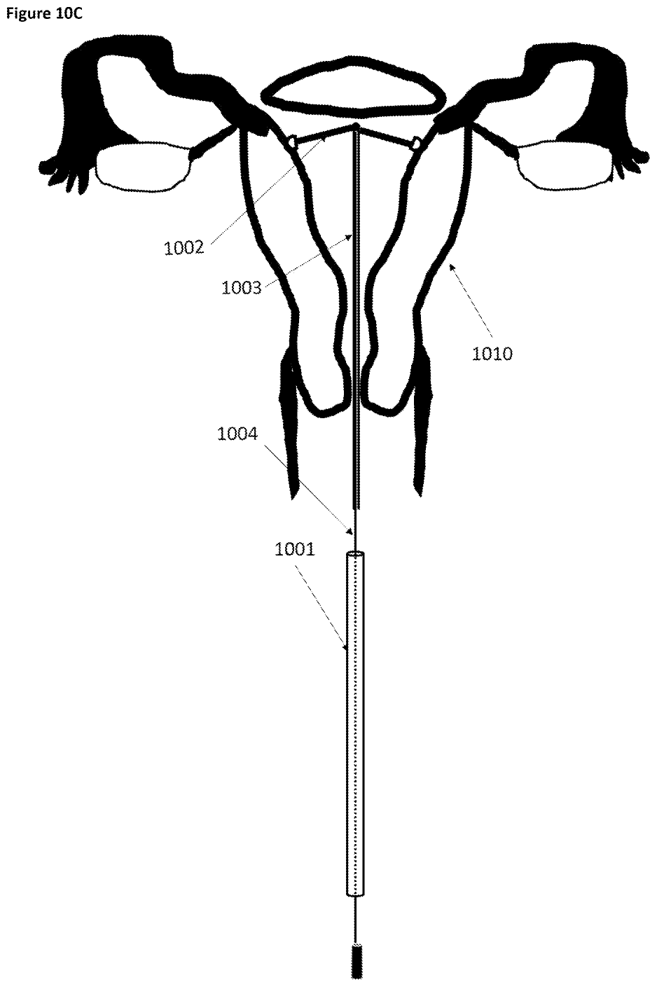

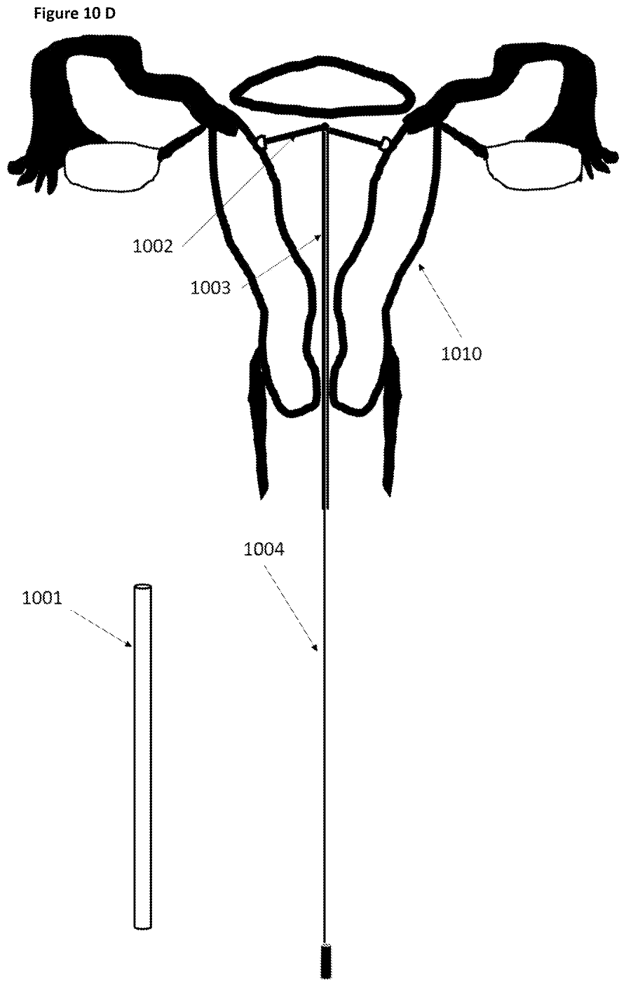

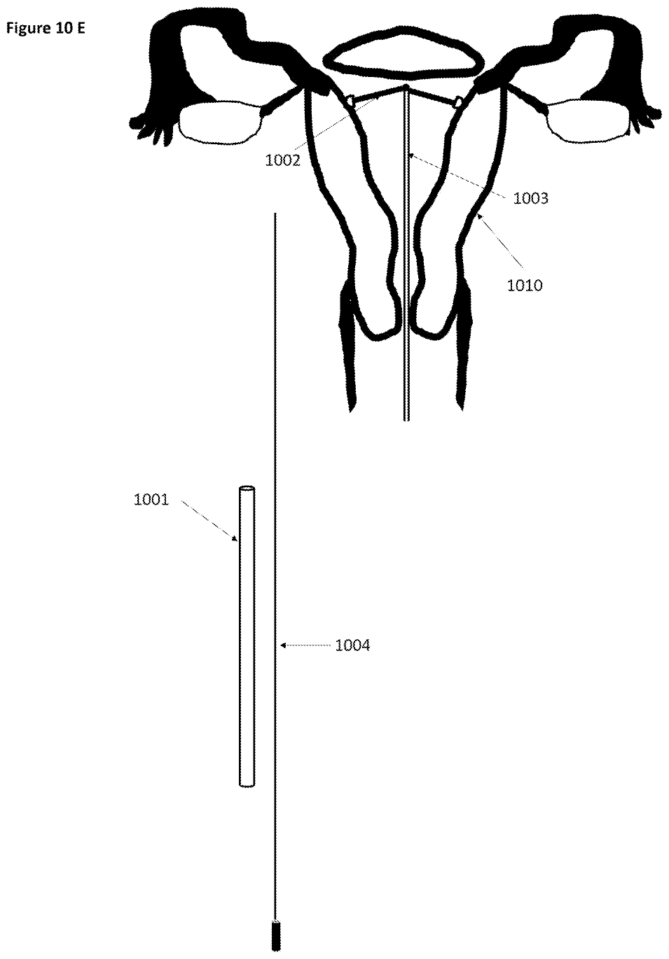

FIG. 10A shows a side view of the exemplary operation of the HDR intra uterine device shown in FIG. 9 with the wings (1002) in a closed configuration when inserted into the uterus (1010), according to an exemplary embodiment of the present disclosure. In particular, the wings (1002) can be housed in the “inserter” (1001). FIGS. 10B-10E illustrate side views of the exemplary operation of the HDR intra uterine device shown in FIG. 9 with the wings (1002) in a configuration when inserted into the uterus (1010), and the progress of the inserter (1001) and a guide wire (1004) being removed from the uterus (1010), according to an exemplary embodiment of the present disclosure.

In particular, during the insertion as provided in FIGS. 10A-10D, the guide wire (1004) is inserted into the HDR channel (1003). After inserting the channel (1003) with the wings (1002) housed in the inserter (1001) into the uterus (1010) (as shown in FIG. 10A), the inserter (1001) can be retracted to facilitate the wings (1002) to open (as shown in FIGS. 10B and 10C). The inserter (1001) can then be fully removed (as shown in FIG. 10D), then the guide wire (1004) can be fully removed (as illustrated in FIG. 10E).

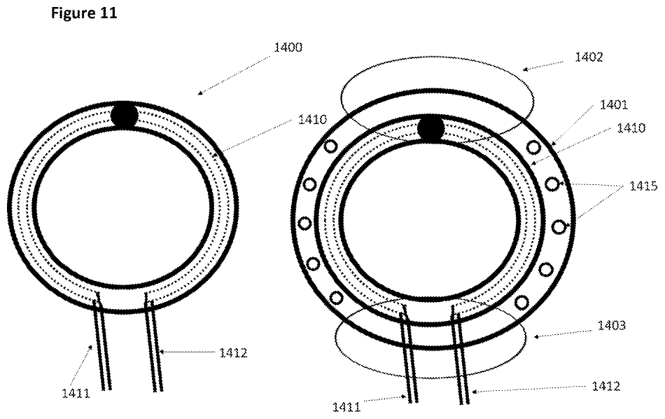

FIG. 11 shows a set of side views of the cervical ring (1400) and (1401), with right and left HDR channels (1411) and (1412), and provided with or without balloons (1402) and (1403) and marked interstitial brachytherapy holes (1415), according to still another exemplary embodiment of the present disclosure. For example, as shown in the left side view of FIG. 11, the balloon (1402) provided at the anterior part of the ring (1401) that increases the distance between the radioactive source and the urinary bladder once inflated. The balloon (1403) at the posterior part of the ring (1401) that increases the distance between the radioactive source and the rectum once inflated. An inner ring can be provided with HDR channel (1410). Marked holes (1415) for insertion of needles can be provided for interstitial brachytherapy.

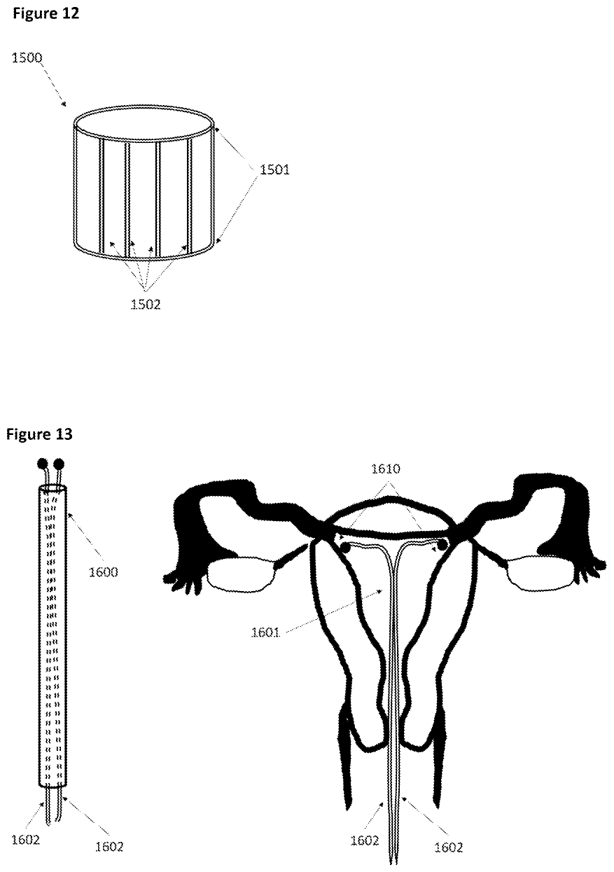

FIG. 12 illustrates a perspective view of a sleeve shaped applicator (1500) that includes proximal and distal rings (1501) with vertical HDR channels (1502) therein, according to a further exemplary embodiment of the present disclosure. The HDR channels (1502) are vertical with respect to the rings (1501), and parallel to the cervix within the sleeve shaped applicator (1500).

FIG. 13 shows a set of views providing a double tandem uterine HDR applicator (1601), and an exemplary operation thereof, according yet another exemplary embodiment of the present disclosure. For example, the double tandem uterine HDR applicator (1601) within an inserter (1600), before insertion guide wires are introduced into the HDR channels (not shown in FIG. 13). The applicator can have two or more HDR channels (1602), and the channels (1602) can have portions thereof, at the innermost part of the uterus, provided in a bent shape. Before insertion, the bent shaped part is forced into the inserter (1600), and when being provided in the uterus, the inserter (1600) is removed, which opens the bent portion of the HDR applicator (1601) and positions each of the HDR channels (1602) in the lateral horns of the uterus, facilitating its fixation in place. This facilitates a delivery of radiation to the center of the uterus and to the uterine horns (1610).

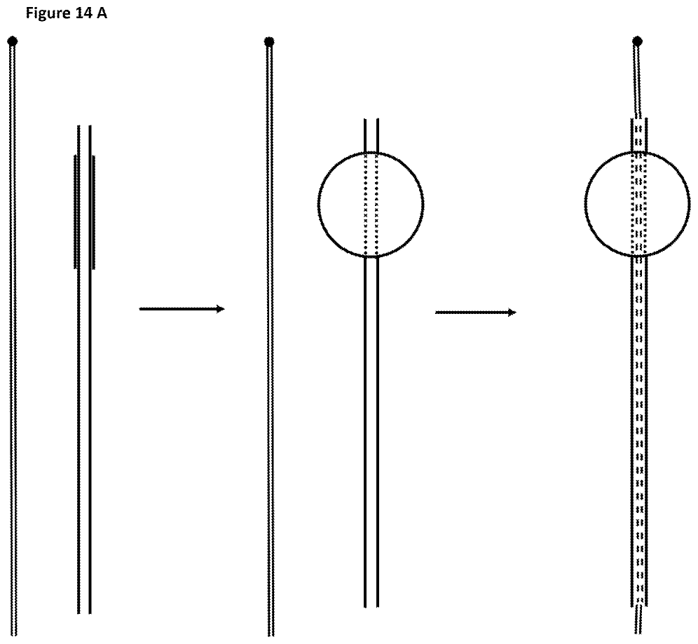

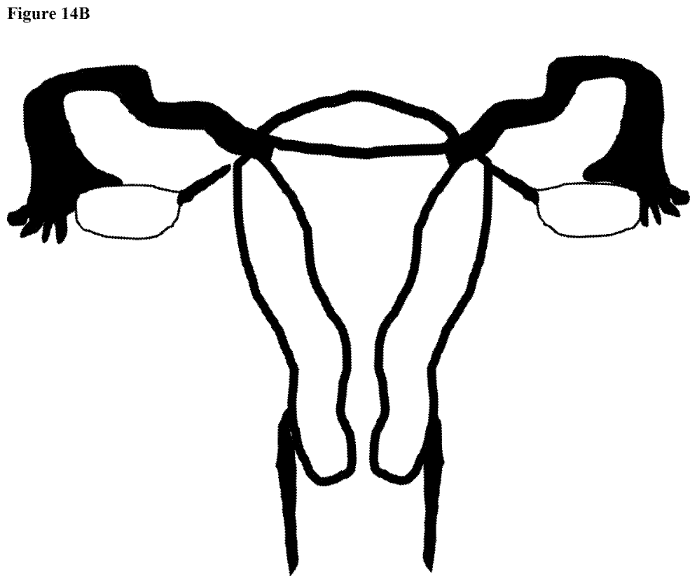

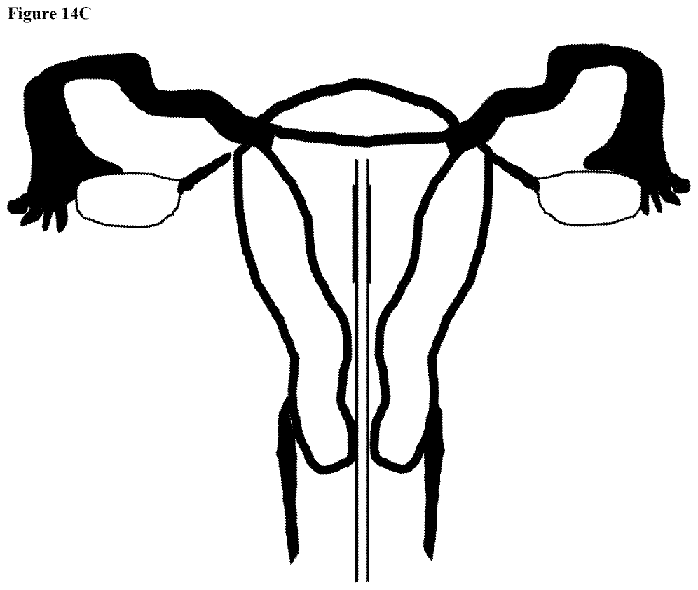

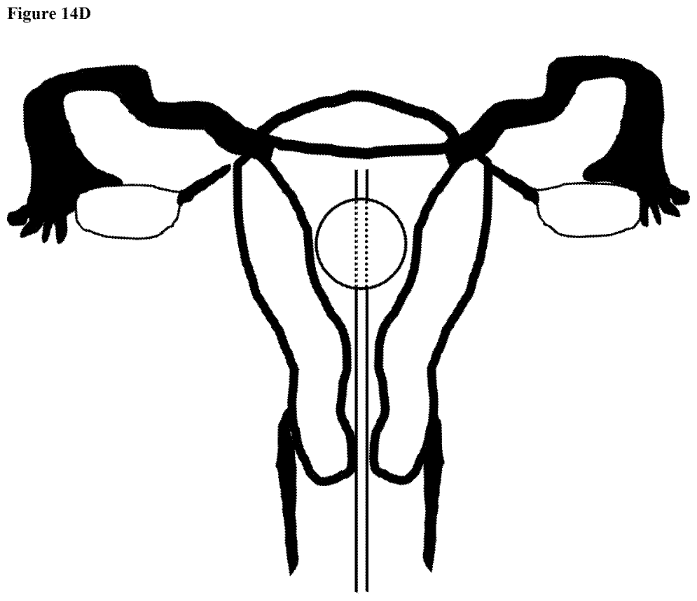

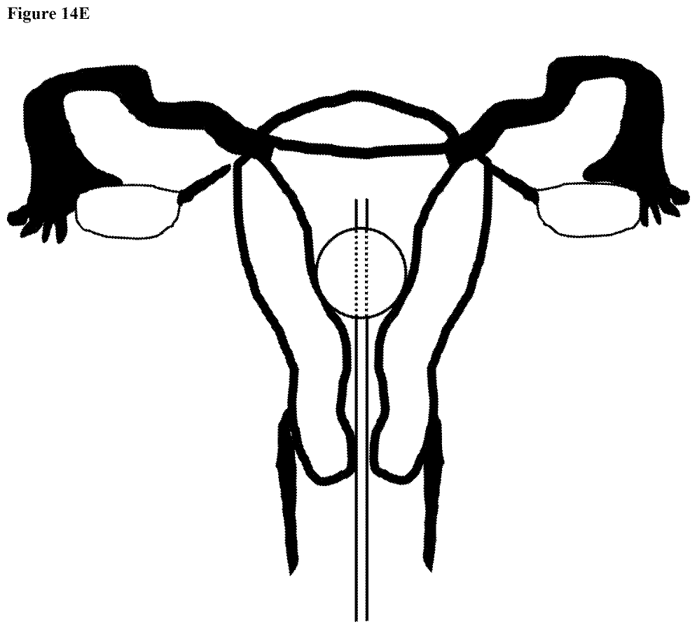

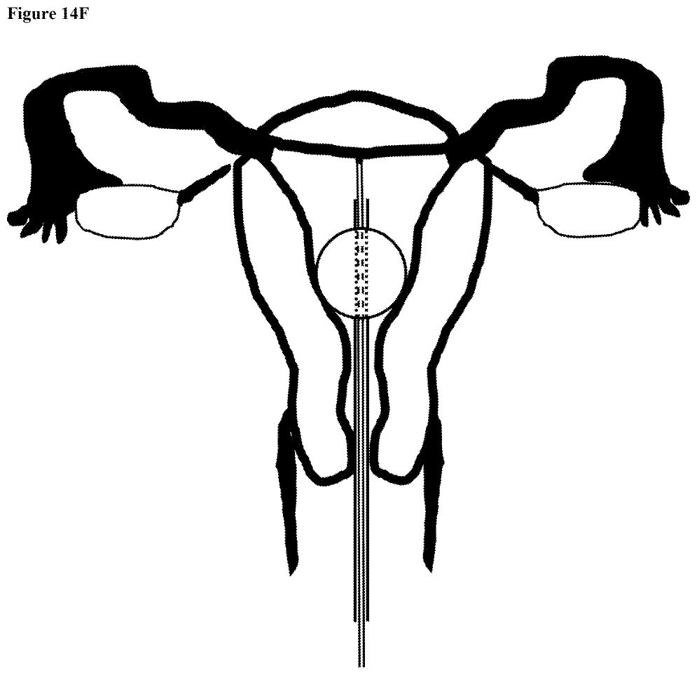

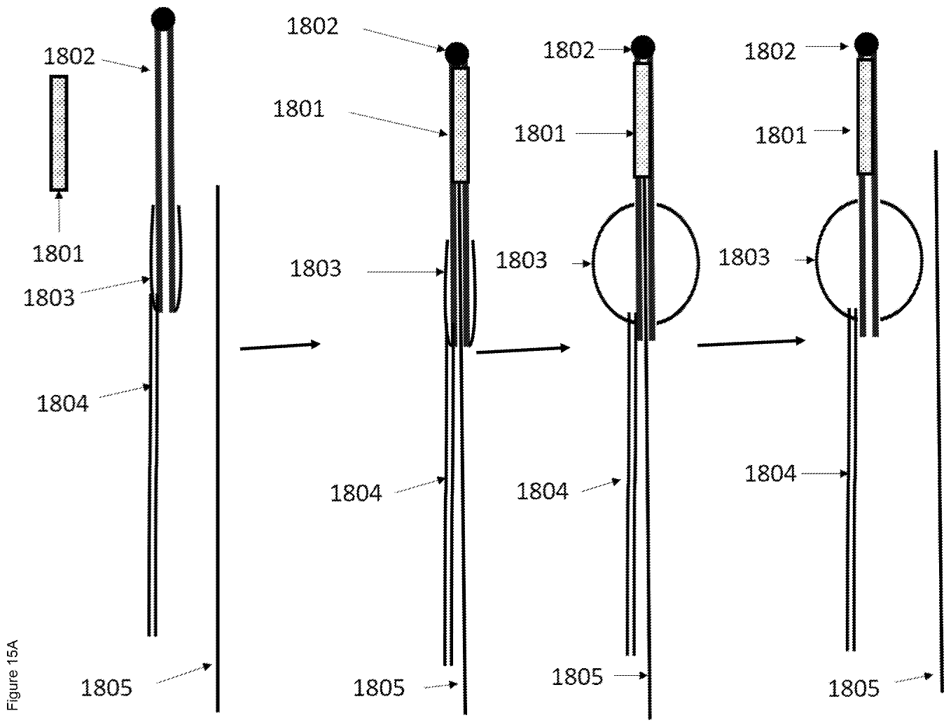

FIGS. 14A-14F illustrate views of the uterine applicator that includes channel(s) which has an inflation unit, and an exemplary operation thereof, according yet a further exemplary embodiment of the present disclosure. As shown in FIG. 14A, a channel that has an inflation unit. Inside the channel, a HDR channel can be inserted. FIG. 14B illustrates the uterus without any device, and FIG. 14C shows a channel with a deflated inflation unit is inserted into the uterus. Then, in FIG. 14D, the inflation unit is inflated. When the inflation unit is inflated, the channel can be gently drawn inferiorly, so the inflation unit anchor the channel to the uterus, as provided in FIG. 14E. A stopper can then be attached to the outer side of the channel (not shown). Further, as shown in FIG. 14F, an HDR channel can be inserted inside the channel, and is advanced to the uterine fundus.

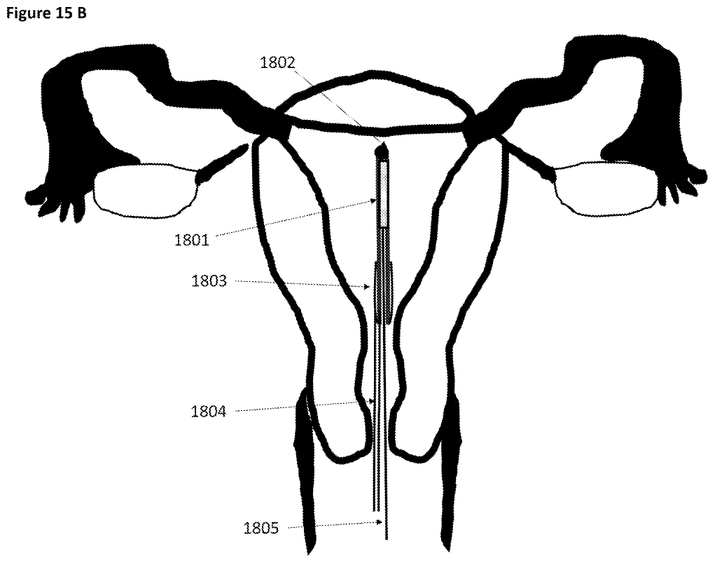

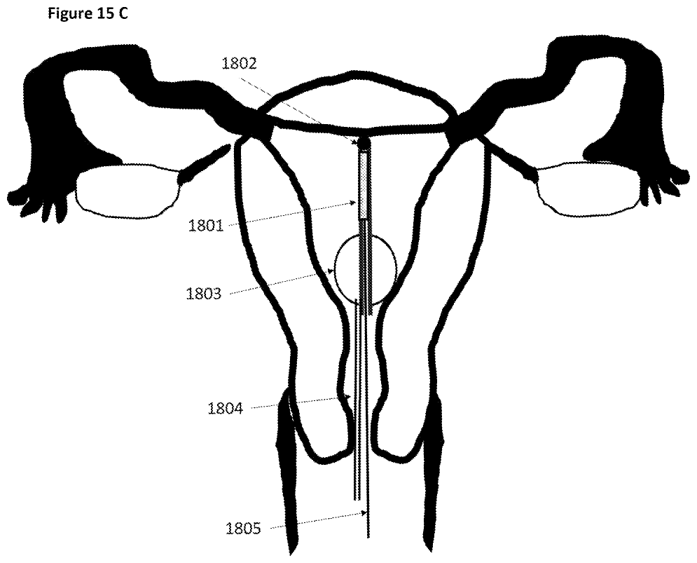

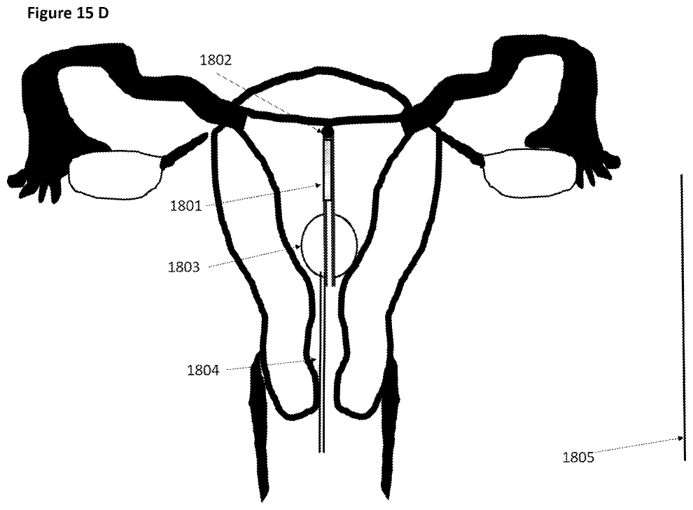

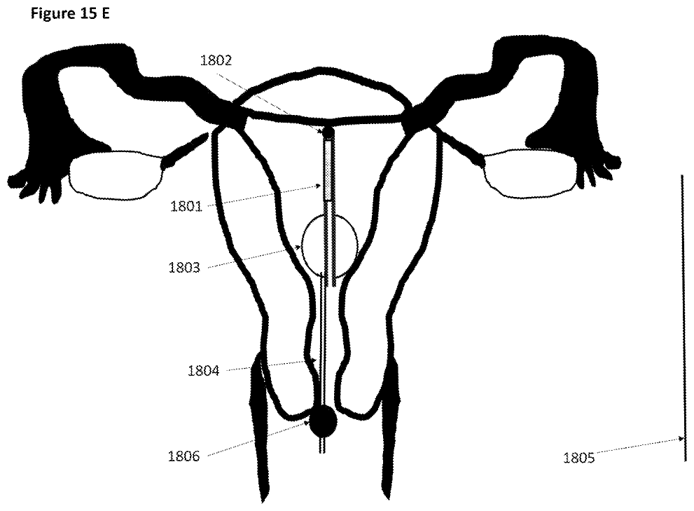

FIG. 15A illustrates a set of side views of an exemplary operation of the contraception intrauterine device for a contraception, according to the exemplary embodiment of the present disclosure. As shown in FIG. 15A, a hormonal formulation (1801), a blunt non sharp end, which can be a balloon to prevent a penetration of the uterus (1802), an inserter channel (1803) with inflation balloon around it, an inflating channel (1804) for inflating the balloon, an inserter rod (1805), and a plug (1806) for blocking the inflating channel (1804) are all illustrated. FIGS. 15B-15E show details of the operation shown in FIG. 15A within the uterus.

In particular, FIG. 15B illustrates the exemplary operation of the contraception intrauterine device inserted into the uterus while the balloon is deflated. FIG. 15C shows a side view of the exemplary operation of the contraception intrauterine device inserted into the uterus and the inflation unit inflated therein. FIG. 15D shows the exemplary operation of the contraception intrauterine device inserted into the uterus and the inflation unit inflated therein and the inserter being removed. FIG. 15E shows the exemplary operation of the contraception intrauterine device inserted into the uterus and inflated therein, the inserter channel removed, and the inflating channel plugged with the plug (1806).

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### Exemplary References:

1. Siegel, R. L.; Miller, K. D.; Fuchs, H. E.; Jemal, A. Cancer Statistics, 2021. CA: A Cancer Journal for Clinicians 2021, 71, 7-33, doi:https://doi.org/10.3322/caac.21654.
2. Key Statistics for Cervical Cancer. Availabe online: https://www.cancer.org/cancer/cervical-cancer/about/key-statistics.html (accessed on 5/6/2021).
3. Cervical cancer. Availabe online: https://www.who.int/health-topics/cervical-cancer#tab=tab_1 (accessed on 5/6/2021).

What is claimed is:

1. A method for providing brachytherapy so as to treat cervical cancer, comprising
inserting an applicator into a uterus of a patient so that it is fully provided in the uterus, the applicator comprising (a) at least one channel for providing at least one radioactive seed and extending outside of the uterus, and (b) a fixation device which is configured to be fixed within the uterus;
fixing the fixation device of the applicator within the uterus without piercing a wall of the uterus so that the applicator and the at least one channel are fixed therein; and
providing the at least one radiation seed into the at least one channel to reach one or more locations with the at least one channel so as to irradiate at least one portion of the uterus and treat the cervical cancer.

2. The method according to claim 1, wherein the fixation device includes at least one of (i) extendable wings, or (ii) at least one balloon.

3. The method according to claim 2, wherein the fixating comprises pulling the balloon downward to impact a lower part of the uterus, and thereby fixing the applicator in the uterus.

4. The method according to claim 1, wherein the applicator includes a sleeve which houses at least two wings therein, and wherein the fixating of the fixation device comprises at least partially removing the sleeve from the uterus so that the wings are exposed and extend outward into the uterus.

5. The method according to claim 1, further comprising:
attaching a ring on a cervix of the patient, wherein the ring includes at least one further channel therein to accept the at least one radiation seed.

6. The method according to claim 5, wherein the ring comprises openings for fitting one or more needles therethrough, and further comprising:
providing at least one further radiation seed via the one or more needles.

7. The method according to claim 1, wherein the at least one channel is connected to a high dose rate (HDR) afterloader, and wherein the HDR afterloader provides the at least one radiation seed to the at least one channel.

8. The method according to claim 1, wherein the applicator includes a stopper at a distal end thereof, and wherein the stopper is configured to reduce or prevent movement of the applicator within the uterus.

9. A device for providing brachytherapy so as to treat cervical cancer, comprising:
an applicator configured to be inserted into a uterus of a patient so that it is fully provided in the uterus,
wherein the applicator comprises (i) at least one channel configured to provide at least one radioactive seed and dimensioned to extend outside of the uterus, and (i) a fixation device which is configured to be fixed within the uterus,
wherein the fixation device of the applicator has a configuration and a size to be fixed within the uterus without piercing a wall of the uterus so that the applicator and the at least one channel are fixed therein, and
wherein the at least one channel is configured and sized so as to provide the at least one radiation seed into the at least one channel to reach one or more locations within the at least one channel so as the at least one radiation seed is facilitated to irradiate at least one portion of the uterus and treat the cervical cancer.

10. The device according to claim 9, wherein the fixation device includes at least one of (i) extendable wings, or (ii) at least one balloon.

11. The device according to claim 10, wherein, in operation, the balloon is configured and sized to be pulled downward to impact a lower part of the uterus, and thereby fixing the applicator in the uterus.

12. The device according to claim 9, wherein the applicator includes a sleeve which houses at least two wings therein, and wherein the fixating of the fixation device comprises at least partially removing the sleeve from the uterus so that the wings are exposed and extend outward into the uterus.

13. The device according to claim 9, further comprising:
a ring configured and sized to be provided on a cervix of the patient, wherein the ring includes at least one further channel therein to accept the at least on radiation seed.

14. The device according to claim 13, wherein the ring comprises openings for fitting one or more needles therethrough, and wherein the one or more needles are configured and sized to provide at least one further radiation seed therein or therethrough.

15. The device according to claim 9, wherein the at least one channel is connected to a high dose rate (HDR) afterloader, and wherein the HDR afterloader is configured to provide the at least one radiation seed to the at least one channel.

16. The device according to claim 9, wherein the applicator includes a stopper at a distal end thereof, and wherein the stopper is configured to reduce or prevent movement of the applicator within the uterus.

17. A method for providing brachytherapy so as to treat uterine cancer, comprising
inserting an applicator into a uterus of a patient so that it is fully provided in the uterus, the applicator comprising (a) at least two channels for providing at least one radioactive seed and extending outside of the uterus, and (b) a fixation device which is configured to be fixed within the uterus;
fixing the fixation device of the applicator within the uterus without piercing a wall of the uterus so that the applicator and the channels are fixed therein; and
providing connection to a High-Dose Rate (HDR) afterloader to facilitate a passing of the at least one radiation seed into the channels to reach one or more locations within the channels;
causing an irradiation of at least one portion of the uterus via the at least one radiation seed so as to treat the uterine cancer.

18. The method according to claim 17, wherein the applicator includes a sleeve which houses at least two wings therein, and wherein the fixating of the fixation device comprises at least partially removing the sleeve from the uterus so that the wings are exposed and extend outward into the uterus.

19. An intrauterine device for a contraception, comprising:
a reservoir housing at least one hormonal compound;
a guidewire provided within an insertion channel; and
at least one inflation unit, wherein, after an insertion of the intrauterine device into a uterus, the at least one inflation unit is caused to be inflated thereby securing the intrauterine device in the uterus, followed by a removal of the guidewire from the insertion channel.

20. An intrauterine device for contraception, comprising:
a reservoir housing at least one hormonal compound;
at least two wings;
at least one channel; and
a guidewire provided with the at least one channel,
wherein, in operation and prior to an insertion of the intrauterine device into a uterus, the wings are secured in a closed configuration via a rope or a cord that passes through said the at least one channel, wherein after the insertion of the intrauterine device into the uterus, the rope or the cord is relaxed which facilitates the wings to open to be in an open configuration, and wherein, when the wings are in the open configuration, the guidewire is removed from the at least channel.
